SEQUENCE LISTING (16 sequences)

SEQ ID NO: 1 - protein, 16 aa, Artificial Sequence
  Synthetic citrullinated peptide
VYATXSSAVXLXSSVP

SEQ ID NO: 2 - protein, 16 aa, Artificial Sequence
  Synthetic peptide
VYATRSSAVRLRSSVP

SEQ ID NO: 3 - protein, 17 aa, Artificial Sequence
  Synthetic citrullinated peptide
NEEGFFSAXGHRPLDKK

SEQ ID NO: 4 - protein, 17 aa, Artificial Sequence
  Synthetic peptide
NEEGFFSARGHRPLDKK

SEQ ID NO: 5 - protein, 21 aa, Artificial Sequence
  Synthetic citrullinated peptide
HQCHQESTXGRSRGRCGRSGS

SEQ ID NO: 6 - protein, 21 aa, Artificial Sequence
  Synthetic peptide
HQCHQESTRGRSRGRCGRSGS

SEQ ID NO: 7 - protein, 15 aa, Artificial Sequence
  Synthetic citrullinated peptide
GPXVVEXHQSACKDS

SEQ ID NO: 8 - protein, 15 aa, Artificial Sequence
  Synthetic peptide
GPRVVERHQSACKDS

SEQ ID NO: 9 - protein, 17 aa, Artificial Sequence
  Synthetic citrullinated peptide
KIHAXEIFDSXGNPTVE

SEQ ID NO: 10 - protein, 17 aa, Artificial Sequence
  Synthetic peptide
KIHAREIFDSRGNPTVE

SEQ ID NO: 11 - protein, 18 aa, Artificial Sequence
  Synthetic citrullinated peptide
GGVYATXSSAVXLXSSVP

SEQ ID NO: 12 - protein, 18 aa, Artificial Sequence
  Synthetic citrullinated peptide
GGVYATXSSAVRLXSSVP

SEQ ID NO: 13 - protein, 18 aa, Artificial Sequence
  Synthetic citrullinated peptide
GGVYATRSSAVXLXSSVP

SEQ ID NO: 14 - protein, 18 aa, Artificial Sequence
  Synthetic citrullinated peptide
GGVYATRSSAVXLRSSVP

SEQ ID NO: 15 - protein, 18 aa, Artificial Sequence
  Synthetic citrullinated peptide
GGVXGPXVVEXHQSACKD

SEQ ID NO: 16 - protein, 18 aa, Artificial Sequence
  Synthetic citrullinated peptide
HSTKXGHAKSXPVXDCDD

(19) United States
(12) Patent Application Publication
(10) Pub. No.: US 2014/0227303 A1
(43) Pub. Date: Aug. 14, 2014

(54) METHODS FOR DIAGNOSING AND MONITORING DISEASES OR CONDITIONS USING DISEASE MODIFIED BIOMOLECULES AND MEASUREMENT OF A FUNCTIONAL IMMUNE RESPONSE

(71) Applicants: Jianrong Lou, Boyds, MD (US); Brad L. Stewart, Gaithersburg, MD (US); Nathaniel Rill, Westminster, MD (US)

(72) Inventors: Jianrong Lou, Boyds, MD (US); Brad L. Stewart, Gaithersburg, MD (US); Nathaniel Rill, Westminster, MD (US)

(73) Assignee: VIRACOR-IBT LABORATORIES, Lee's Summit, MO (US)

(21) Appl. No.: 14/129,445

(22) PCT Filed: Jun. 29, 2012
(86) PCT No.: PCT/US2012/044858

§ 371 Date: Apr. 16, 2014

Related U.S. Application Data
(60) Provisional application No. 61/502,448, filed on Jun. 29, 2011.

(51) Int. Cl. G01N 33/569

(52) U.S. Cl. CPC G01N 33/56966
USPC 424/185.1; 435/7.1

(57) ABSTRACT

Methods and assays for disease prognosis, detection and treatment monitoring are provided. The assays and methods measure functional immune responses to disease modified biomolecules (DMBs) that are characteristic of a disease of interest. Exemplary diseases of interest include rheumatoid arthritis (RA), for which citruUinated peptides or proteins (CPs), are characteristic DMBs.

### BACKGROUND OF THE INVENTION

1. Field of the Invention

The invention generally relates to disease prognosis, detection and/or treatment monitoring. In particular, the invention provides assays and methods which measure functional immune response(s) to disease modified biomolecules (DMBs) that are characteristic of a disease of interest.

2. Background of the Invention

The detection of disease at an early stage is a desideratum in the medical field, as are the confirmation of the presence of disease, and the ability to monitor the progress of disease and the efficacy of therapeutic treatments. Detection and confirmation of disease at an early stage and the ability to accurately and systematically monitor disease status thereafter often result in improved clinical outcomes. Unfortunately, in many instances, it is necessary for overt disease symptoms to develop before a diagnosis can be made or confirmed, and observation of gross symptoms is currently often the only practical way to determine the efficacy of treatment. However, by the time overt symptoms appear, irreversible damage to a subject's health may have already taken place. In addition, readily observable symptoms are often common to many different diseases, and even some detectable disease “biomarkers” have been found to be associated with more than one type of diseases, and/or to be present in apparently healthy individuals. These factors negatively impact health care professionals' ability to provide reliable, specific diagnoses at early stages of a disease, and hamper their ability to successfully provide early intervention and to make successful adjustments to treatment protocols.

Cancer, cardiovascular disease, diabetes, progressive neurological disorders, infectious diseases and autoimmune diseases such as rheumatoid arthritis are all examples of disorders where early detection, confirmation and monitoring are highly desirable. For example, as discussed in detail below, the autoimmune disease rheumatoid arthritis (RA) is frequently not reliably diagnosed until after irreversible joint damage has already occurred. The most frequent blood test used in the diagnosis of RA is the measurement of rheumatoid factor (RF). RF is an autoantibody against the constant region of immunoglobulins of the IgG subclass, the presence of which is easily detected in plasma samples by immunological methods. While RF can be detected in about 50-80% of RA patients, RF is not detectable in all RA patients. In addition, RF can also be detected in high percentages of individuals with other inflammation conditions, as well as in some apparently healthy individuals. Thus, although the assay for detecting RF has relatively good sensitivity, the lack of specificity limits its clinical applications.

For a wide variety of diseases, including autoimmune diseases such as RA, the prior art has thus-far failed to provide specific, sensitive, reliable methods for predicting, diagnosing, and monitoring disease progress and/or treatment regimens. In particular, the prior art has not provided methods or assays to diagnose many diseases at a very early stage, e.g., prior to the development of overt disease symptoms, thereby enabling early intervention.

### SUMMARY OF THE INVENTION

The present invention provides methods for rapidly and sensitively predicting, diagnosing, and/or monitoring treatment regimens of many diseases, including autoimmune diseases such as RA. This is accomplished by determining whether or not the immune cells of a subject are responsive to a disease modified biomolecule (DMB) which is produced when a particular disease state is present in a subject. A DMB is a molecule that is not typically found in a subject without the disease or condition of interest. The physiological basis for this is depicted in FIG. 1, which provides a schematic representation of the development of primed immune cells in a subject and their detection using the methods and assays of the present invention.

With reference to FIG. 1, the first rectangle represents a subject and the enclosed ovals represent immune cells of the subject which have never been exposed to a DMB. When a disease develops in the subject, conditions within the subject's body change and as a result DMBs are produced, e.g., by post-translational modification of proteins or peptides, or in other ways. Immune cells in the subject are exposed to the DMBs, and the DMBs act as antigens, causing activation of the body's immune system including innate and/or adaptive immunity. On the other hand, if no DMB is present in the subject, then the immune cells are not activated or primed. The presence of DMBs can either precede or succeed the onset of a particular disease. Both genetic and environmental factors may play significant roles in triggering activation of the immune system.

To practice the methods of the invention, a sample of immune cells from a subject is obtained. Once a sample is obtained, DMBs are added to the sample, thereby exposing the immune cells in the sample to the DMBs. If the immune cells are not primed with respect to the DMBs, i.e., if they have not been previously exposed to the DMBs, or if they are not capable of mounting a response, then they will not react to the DMBs in a manner that is detected by the present methods. However, if the immune cells are already activated with respect to the DMBs, i.e., if they had been previously exposed to the DMBs, then they do react to the presence of the DMBs. The reaction of previously activated immune cells is to produce one or more different substances associated with an immune response, referred to in FIG. 1 as an “indicator substance”. Thus, the final step of the method is to detect whether or not indicator substances are present in the sample. If an indicator substance is detected, then one practicing the method would conclude that the subject likely has or is in the process of developing the disease of interest. On the other hand, if indicator substances are not detected in the sample, or if a level of indicator substance is detected that is not significantly different from that which is found in healthy populations, then one practicing the method would conclude that the subject does not have the disease or that the disease is controlled or in remission.

The methods and assays of the invention can also be used to monitor the efficacy of disease treatments. If treatment progresses successfully, decreased immune response is detected in the assay. However, if treatment is not working as desired, then the results of the assay may remain constant, or not decrease as rapidly or as greatly as expected, or the amount of indicator substance that is detected may even increase if the disease progresses.

It is an object of this invention to provide methods and assays for screening, diagnosing, prognosing, and/or predicting disease, and/or for monitoring the response of a patient to disease therapy. In some embodiments, the methods and assays are used to determine which drug(s) should be used for therapy, and/or whether a therapy protocol should be changed, e.g., by switching or changing the dose of one or more drugs. The methods and assays may be used to provide assessments that are quantitative or qualitative, or both, in nature. The methods and assays may also be used for longitudinal studies or assessments over time, both for individual patients and for groups of patients, e.g., patients or subjects enrolled in clinical trials for testing the efficacy of a treatment.

It is an object of the invention to provide a method of determining whether or not a subject has or is at risk of developing a disease or condition of interest. The method comprises the steps of 1) obtaining at least one disease modified biomolecule (DMB) that is identified as being present in biological samples from one or more subjects in a pool of subjects with the disease of interest, wherein the DMB does not appear or the DMB level is significantly lower (e.g., statistically significantly lower relative to an appropriate control)) in biological samples from one or more control subjects who do not have the disease or condition of interest; 2) obtaining a biological sample from a subject that has or may have the disease or condition of interest; 3) exposing the biological sample or cells from the biological sample to the DMB; 4) detecting at least one functional immunological response in the biological sample; and, if at least one functional immunological response is detected, then 5) concluding that the subject has or is at risk of developing the disease or condition of interest. In one embodiment, the DMB is present in at least 50% of biological samples obtained from said pool of subjects. In some embodiments, the step of concluding includes a step of comparing results obtained in said detecting step with results obtained in at least one of: i) control subjects who do not have the disease or condition of interest; ii) control subject who do have the disease or condition of interest; iii) patients with the disease or condition of interest who are successfully responding to treatment; and iv) patients who are positive for the presence of the at least one DMB but who have not developed other symptoms of the disease or condition of interest. In other embodiments, the at least one functional immunological response is selected from the group consisting of: production of expression products of one or a plurality of genes; production of one or a plurality of mRNA molecules; production of one or a plurality of microRNAs (miRNAs); production of one or a plurality of proteins; and production of one or more of ATP, cytokines, interferon-gamma (IFN-γ), glucose, and nicotinamide adenine dinucleotide (NADH). In yet other embodiments, the disease or condition of interest is rheumatoid arthritis (RA) and the DMB is a citrullinated peptide or protein (CP). For example, the DMB may be a CP that is or comprises a citrullinated amino acid sequence VYAT[Cit]SSAV[Cit]L[Cit]SSVP (SEQ ID NO: 1), or a functional variant thereof; or a citrullinated amino acid sequence GGVYAT[Cit]SSAV[Cit]L[Cit]SSVP (SEQ ID NO: 11), or a functional variant thereof.

The invention further provides a method of monitoring the efficacy of treatment of a disease or condition of interest in a patient in need thereof. The method comprises the steps of i) obtaining at least one disease modified biomolecule (DMB) that is identified as being present in biological samples from one or more subjects in a pool of subjects with the disease of interest, wherein the DMB does not appear or the DMB level is significantly lower in biological samples from one or more control subjects who do not have the disease or condition of interest; ii) obtaining a biological sample from a subject that has and is receiving treatment for the disease or condition of interest; iii) exposing the biological sample or cells from the biological sample to the DMB; iv) detecting and/or quantifying at least one functional immunological response in the biological sample; and, based on results obtained in said detecting and/or quantifying step v) determining whether or not said treatment is efficacious. In some embodiments, the DMB is present in at least 50% of biological samples obtained from the pool of subjects. In other embodiments, the method also comprises the steps of repeating steps i)-iv) for said patient at each of a plurality of successive, spaced-apart time intervals; and comparing results obtained at said plurality of successive, spaced-apart time intervals in order to determine whether or not said treatment is efficacious. In yet other embodiments, the at least one functional immunological response is selected from the group consisting of: production of expression products of one or a plurality of genes; production of one or a plurality of mRNA molecules; production of one or a plurality of microRNAs (miRNAs); production of one or a plurality of proteins; and production of one or more of ATP, cytokines, interferon-gamma (IFN-γ), glucose, and nicotinamide adenine dinucleotide (NADH). In some embodiments of the invention, the disease or condition of interest is rheumatoid arthritis (RA) and the DMB is a citrullinated peptide or protein (CP). Exemplary CPs are or comprise a citrullinated amino acid sequence VYAT[Cit]SSAV[Cit]L[Cit]SSVP (SEQ ID NO: 1), or a functional variant thereof; and/or a citrullinated amino acid sequence GGVYAT[Cit]SSAV[Cit]L[Cit]SSVP (SEQ ID NO: 11), or a functional variant thereof.

The invention further provides a composition comprising a citrullinated peptide or protein comprising one or both of i) a citrullinated amino acid sequence VYAT[Cit]SSAV[Cit]L[Cit]SSVP (SEQ ID NO: 1) or a functional variant thereof, and ii) a citrullinated amino acid sequence GGVYAT[Cit]SSAV[Cit]L[Cit]SSVP (SEQ ID NO: 11) or a functional variant thereof; and a physiologically acceptable carrier. In some embodiments, the citrullinated peptide is or comprises VYAT[Cit]SSAV[Cit]L[Cit]SSVP (SEQ ID NO: 1) or a functional variant thereof. In other embodiments, the citrullinated peptide is or comprises GGVYAT[Cit]SSAV[Cit]L[Cit]SSVP (SEQ ID NO: 11) or a functional variant thereof.

### BRIEF DESCRIPTION OF THE DRAWINGS

FIG. 1. Schematic representation of the origin of DMBs and the in vitro detection of immune cells which have been primed as a result of prior in vivo exposure to DMBs.

FIG. 2. Schematic representation of the assay of the invention.

FIG. 3. IL6 concentrations in culture supernatants stimulated by peptide DMB 1011 to DMB 1016.

FIG. 4. TNFα concentrations in culture supernatants stimulated by peptide DMB 1011 to DMB 1016.

FIG. 5. IL6 concentration after DMB 1001 peptide stimulation.

FIG. 6. TNFα concentration after DMB 1001 peptide stimulation.

FIG. 7. Cytokine profiles after cell stimulation with DMB 1001.

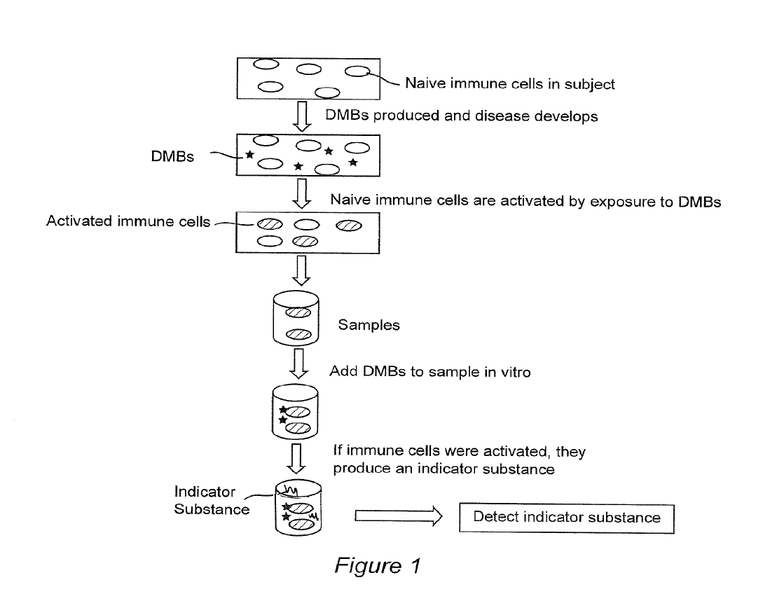

### DETAILED DESCRIPTION

The invention provides methods and assays for interrogating the immune status of an individual with respect to a particular modified molecule (or molecules) that is/are associated with or characteristic of a particular disease or condition of interest, i.e., a “disease modified biomolecule” or “DMB”. In one embodiment, the disease or condition of interest is an autoimmune disease. The methods and assays detect, in vitro, whether or not cells in the immune system of an individual are responsive, in vivo, to a DMB that is characteristic of a particular disease or condition of interest. The DMBs are biomolecules that are somehow changed (e.g., chemically or structurally modified) as a result of the presence of disease or incipient disease or condition in an individual. The disease-specific modifications of the biomolecule do not occur in disease-free individuals. Therefore, when immune cells of a disease-free individual are tested in vitro with respect to immune activation, the response will be similar to background levels, since activation is not present. In contrast, when an individual has the disease or condition of interest (whether or not overt symptoms have appeared), disease conditions in the patient will have resulted in the production of DMBs that are characteristic of or specific to the disease or condition of interest and activation of immune cells will have occurred. Therefore, when immune cells of an individual with the disease or condition of interest are tested in vitro with respect to prior activation, the response will be positive since prior exposure, and hence activation, will have occurred.

In order to carry out the methods and assays of the invention, DMBs associated with particular diseases or conditions of interest are obtained. In some embodiments, DMBs are identified de novo as described herein. In other embodiments, the DMBs may be those which are well recognized in the art as being characteristic of a disease or condition of interest. Exemplary DMBs include but are not limited to: CPs (citrullinated peptides or proteins) which are associated with RA; etc. As used herein, “DMB” or “DMBs” may, in some embodiments, refer to a single molecule with a specific chemical formula. However, in other embodiments, these terms may relate to a plurality of molecules with differing chemical structures but which each have a particular type of chemical modification. An example of the former would be one particular amino acid residue which has a particular, defined chemical modification. An example of the latter would be a collection of amino acids or peptides which differ from one another, e.g., dipeptides, tripeptides, peptides with different primary sequences, etc., (although some of the molecules in the group may be the same) but all of which have at least one specific and defined chemical modification in common (e.g., citrullination). Further, more than one type of chemical modification may be associated with one or more DMBs, e.g., a combination of citrullination plus sulfation may occur. Further, the numbers (amount, extent, etc.) of chemical modification may vary within a particular type of DMB, e.g., some RA associated CPs may be heavily citrullinated (e.g., many citrulline groups per peptide), while others may be sparsely citrullinated (e.g., only a few citrulline groups per peptide. Any such type(s) of modified molecule(s) are intended to be encompassed by the terms DMB and DMBs, so long as they are generated in response to disease conditions or other conditions within a subject with a disease or condition of interest, and so long as exposure of the immune cells of the subject to the molecules elicits a cell functional immune response (activates immune cells) that is detectable as described herein.

In one embodiment of the invention, the disease of interest is RA and the DMB is a citrullinated peptide that is, or comprises, the amino acid sequence VYAT[Cit]SSAV[Cit]L[Cit]SSVP (SEQ ID NO: 1), or a functional variant thereof. This sequence represents a peptide motif sequence that has been shown to stimulate PBMCs of RA patients in a specific or selective manner. This sequence, and/or peptides or polypeptides which comprise the sequence, can thus be used in the practice of the invention to detect or diagnose RA, to confirm RA diagnoses, to provide information regarding whether or not an RA patient is or is not likely to respond to therapy (e.g., therapy that involves inhibition of particular cytokines), etc. Exemplary peptides which include this motif include but are not limited to: GGVYAT[Cit]SSAV[Cit]L[Cit]SSVP (SEQ ID NO: 11), or a functional variant thereof.

In another embodiment of the invention, the disease of interest is also rheumatoid arthritis and the associated DMBs are carbamylated peptides, polypeptides and proteins.

In another embodiment of the invention, the disease of interest is Celiac disease and the associated DMBs are deamidated gluten peptides, polypeptides and proteins. Celiac disease is an autoimmune disorder which affects the small intestine and nutrient absorption. About 1% of population in the United States has celiac disease. The symptoms, which usually vary among individuals, include chronic diarrhea, fatigue, loss of weight and abdominal pain. When exposed to gluten, the enzyme transglutaminase (TG2) converts glutamine residues of gluten peptides to glutamate. The deamidated peptides serve as antigens which stimulate the immune system. Detection of the functional immune responses by DMB such as deamidated peptides for celiac disease can be used for diagnosing and monitoring the disease, including disease progression when therapeutic measures are undertaken, e.g., dietary changes, administration of medication, etc.

In another embodiment of the invention, the disease of interest is multiple sclerosis (MS) and the associated DMBs are peptides, polypeptides and proteins based on malondialdehyde modified myelin oligodendrocyte glycoprotein (MOG). MS is an autoimmune disease that involves the central nervous system (CNS). MS affects the ability of nerve cells to communicate with each other effectively usually due to inflammation and its damages to myelin sheath. MOG is the critical component of myelin. The detection of the immune response to post-translation MOG modification products such as, for example, malondialdehyde modified MOG, could be used for disease diagnosis and monitoring as described herein.

In yet another embodiment of the invention, the disease of interest is Type 1 diabetes. Type 1 diabetes (T1D) is a chronic (usually lifelong) autoimmune disease and one of the most prevalent autoimmune disorders among children. Recent statistics showed that T1D incidence is rising about 3% annually. The etiology of T1D is complex and both genetic background and environmental factors contribute the T1D pathogenesis. DMBs associated with T1D include but are not limited to: post translational modification products of insulin and glutamic acid decarboxylase (GAD) isoforms GAD65 and GAD67. One exemplary DMB is insulin A chain in which disulfide bonds have formed between cysteine residues at one or more locations in the polypeptide chain.

Many other diseases, disease states or conditions may also be assessed by the methods and assays disclosed herein, examples of which include but are not limited to: inflammatory bowel disease (e.g., Crohn's disease and ulcerative colitis), cardiovascular disease, systemic lupus erythematosus, diabetes (e.g., Type 1 diabetes, as discussed above), cancer, infectious diseases (e.g., tuberculosis, malaria, influenza, diseases caused by trypanosomes, etc.).

Functional variants of the sequences disclosed herein include peptides that have a least about 75, 80, 85, 90, 95, 96, 97, 98 or 99% identity with the disclosed sequences, and/or which have at least about 75, 80, 85, 90, 95, 96, 97, 98 or 99% similarity with the disclosed sequences, when assessed using an alignment and comparison program, many of which are well known in the art. Such sequences retain the same or a similar functional level as the sequences disclosed herein when used in the assays and methods of the invention, e.g., they are at least about 50, 60, 70, 80, 90, or 100% as active, or may be even more active, e.g., 1, 2, 3, 4, 5, 6, 7, 8, 9, or 10-fold more active. Those of skill in the art will recognize that such functional variants or derivatives may contain, for example, conservative amino acid substitutions, e.g., hydrophobic amino acids such as Ala, Leu and Val may be interchanged and/or replaced by one another; etc. Generally, for citrullinated peptides, the pattern of citrullination is retained in such variants, although this need not always be the case: additional residues may be citrullinated, or some residues that are citrullinated in the disclosed sequences may not be in a variant, the total number of citrullinated residues may remain the same or may vary, etc. Generally, the number of citrullinated residues in a peptide is at least 2, and usually 3 or more (e.g., 4, 5, 6, 7, 8, 9, 10 or more), depending e.g., on the length and sequence of the peptide/polypeptide. In general, at least one residue is citrullinated and at most all residues are citrullinated, with citrullination being possible at any position and at any combination of positions.

Those of skill in the art will recognize that the amino acid sequence set forth as SEQ ID NO: 11 is, in fact, a variant of SEQ ID NO: 1, having an additional two Gly residues at the amino terminus. The presence of the two additional Gly residues increases the water solubility of the peptide. Those of skill in the art will recognize that many similar variants of the peptides disclosed herein can be designed and constructed, e.g., in order to further increase solubility (e.g., water solubility), to increase or decrease hydrohobicity or hydrophlicity, to add targeting sequences (e.g., antibodies or portions thereof, leader sequences, etc.), to add moieties that allow detection of the peptides (e.g., one or more Trp residues for fluorescence detection, or a detectable chemical label), or to facilitate isolation of the peptide (e.g., a His or similar tag), or to prevent or discourage hydrolysis or proteolytic cleavage of the peptides (e.g., acetylation or addition of various alkane or alkyl groups), etc.

In addition, the peptides of the invention may be varied by the incorporation of non-natural amino acids, e.g., D amino acids, carnitine, hydroxyproline, selenomethionine, ornithine, dehydroalanine, 2-isobutyric acid, gamma isobutyric acid, β amino acids such as β-alanine, etc. In addition, one or more residues of the peptides may be chemically modified e.g., by hydroxylation, sulfation, carboxylation, acetylation, and/or by other methods that are known to those skilled in the art. Further, the peptides may contain either conventional or reduced peptide bonds.

Additional variants include but are not limited to, for example: various modifications at the amino and/or carboxy terminus of the peptide. Exemplary embodiments include but are not limited to: the addition of biotin to the peptides in order to adapt them to a system using strepavidin coated beads or plates; linking of fluorescence molecules to the peptides; conjugation of the peptides to other proteins or peptides for co-stimulation, etc.

Those of skill in the art are well acquainted with the preparation of peptides, e.g., by chemical synthesis or recombinant techniques. The peptides disclosed herein may be manufactured by any such suitable means. Likewise, purification methods (e.g., chromatography, etc.) are well known and may be used to substantially purify the peptides. By substantially purified, we mean that the peptides are at least about 90-100% free of contaminating (non-peptide) molecules, with the exception of ions (e.g., Na, Cl, or phosphate ions, etc.), water molecules, etc.

In other embodiments, the invention also encompasses protein preparations which contain the uncitrullinated (non-citrullinated) peptide sequences disclosed herein, e.g., contained within one or more proteins. For example, recombinant proteins (e.g., from bacterial cells) such as vimentin which comprise the disclosed sequences (or another polypeptides or proteins which are genetically engineered to contain and express nucleic acids encoding the peptides sequences) can also be in vitro citrullinized, e.g., by the enzyme PADI (peptidyl-arginine deiminase). The citrullinized proteins would have a similar function to that of the citrullinized peptide(s) and hence may be used in the practice of the invention.

The DMBs of the invention are “associated with” a particular disease or condition of interest. By “associated with” we mean that they are generally only produced in subjects as a result of the presence of a particular disease or condition, although in some cases, a DMB may be associated with more than one disease. The DMBs are thus also described as being “characteristic of” or a “hallmark of” the disease, and if they are associated with only one disease or condition, they may be referred to as “specific for” or “associated specifically with” that disease, or other equivalent expressions. Many diseases, disease states or conditions may be assessed by the methods and assays disclosed herein. Exemplary diseases/conditions include but are not limited to: RA, multiple sclerosis (MS), inflammatory bowel disease, cardiovascular disease, celiac disease, systemic lupus erythematosus, diabetes, cancer, infectious diseases, etc. Any condition or constellation of symptoms that is/are generally recognized by health care professionals as being detrimental to, or potentially detrimental to, the health and well-being and/or longevity and/or quality of life of an individual, may be subject to analysis using the methods and assays described herein.

The DMBs may need to be identified for a particular disease or condition of interest in the practice of this invention. Those of skill in the art are familiar with techniques for identifying previously unknown molecules associated with particular biological phenomena such as diseases, e.g., for identifying new DMBs. Briefly, this is usually accomplished by screening, using known techniques, a number of biological samples from individuals known to have a disease or condition of interest, and comparing the results from the screened samples with results obtained from disease-free individuals (controls). Substances that appear in, for example greater than at least about 50% of the disease samples (or possibly in higher percentages, e.g., about 55, 60, 65, 70, 75, 80, 85, 90, 95, or even 100% of disease samples) may represent a DMB, especially if the substance is generally not detected in healthy, control samples at levels greater than about 50%, preferably about 45, 40, 35, 20, 25, 20, 15, 10, 5 or even less, e.g., none or only trace amounts (e.g., less than about 1% of the disease samples) of the level that is found in disease samples. Alternatively, most control samples may contain the substance of interest, but at a level that is generally consistently lower than is found in disease samples, less than about 50, 45, 40, 35, 20, 25, 20, 15, 10, 5, or 1% of the level that is typically detected in disease samples. In this case, the presence of the DMB in individuals with the disease or condition may be referred to as “elevated”.

Once one or more DMBs is identified, the chemical structures of isolated and purified samples thereof are determined by techniques that are well known to those of skill in the art, e.g., by mass spec, by nuclear magnetic resonance, etc.

Once the chemical structure(s) has/have been determined, pools of isolated and purified DMBs are obtained for use in the methods and assays of the invention. The DMBs may be, for example, isolated from biological samples from persons afflicted with the disease of interest and then purified. Alternatively, DMB may be chemically synthesized. In other embodiments, e.g., when the DMB is a modified peptide or polypeptide, the peptide or polypeptide may be produced either synthetically or using molecular biology techniques (e.g., nucleic acid sequences encoding the peptide or polypeptide may be cloned into an expression vector and desired quantities of the peptide/polypeptide may be produced, and then chemically modified prior to use).

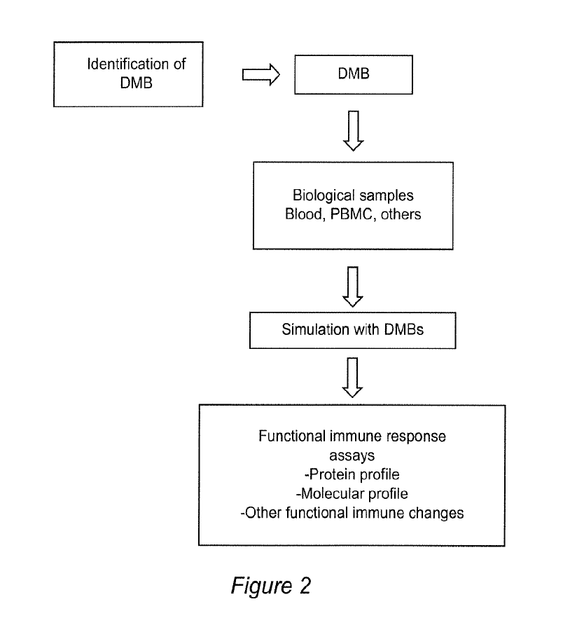

The processes of identification and procurement of DMBs suitable for in vitro assays are depicted schematically at the top of FIG. 2, which shows “identification of DMB” followed by “DMB”, the latter representing the first ingredient in the assay method. Further steps of the assay are also depicted schematically in FIG. 2, and involve the following:

In order to detect prior exposure to a DMB of interest, a biological sample is obtained from a subject who may have or who may develop a disease or condition of interest, and at least one type of immune cell in the sample is exposed to a DMB characteristic of the disease or condition of interest, i.e., the DMBs are used to stimulate the cells in the sample. Those of skill in the art are familiar with obtaining biological samples, which are frequently but not always biological fluids such as blood, serum, synovial fluid, saliva, urine, spinal fluid, etc. However, in some embodiments various tissues may be sampled or be included in the sample, e.g., biopsy tissue samples.

Following stimulation of the cells in the sample with a DMB or a plurality of different DMBs, the response of the immune cells in the sample to the one or more DMBs is monitored, for example, by detecting or measuring at least one change in the cells such as a functional immune response [e.g., a cell mediated immune (“CMI”) response]. The stimulation of immune cells by DMB(s) may or may not require the presence of co-stimulation factors as described elsewhere herein. Such a change may be production of one or more substances known to be associated with previous immune activation, i.e., substances known to be associated with functional immune responses (see FIG. 2). Such substances may include but are not limited to: the expression products of one or a plurality of genes e.g., one or a plurality of mRNA molecules, which may generate a unique, detectable mRNA profile; one or a plurality of microRNAs (miRNAs) may generate a unique, detectable miRNA profile; DNA modification (e.g., methylation, demethylation, histone modification, etc.), one or a plurality of proteins, which may generate a unique, detectable protein profile; one or more substances that are produced by activated immune cells in response to exposure to an activating antigen such as a DMB, e.g., ATP, cytokines (e.g., type 1 (IFN-γ, TGF-β, etc.), and type 2 (IL-4, IL-10, IL-13, etc.), TNF α, IL6, IL 1b, etc.); interferon-gamma (IFN-γ), glucose, nicotinamide adenine dinucleotide (NADH), etc. Those of skill in the art are familiar with techniques for detecting such molecules, e.g., using ELISA, nucleic acid amplification (e.g., PCR), sequencing of peptides/proteins and/or nucleic acids, HPLC analyses, gel electrophoresis and staining, mass spec, etc.

The final step in one embodiment of the method involves interpreting and then drawing a conclusion from the results. If no functional immune response is detected, then the immune elements in the sample are not primed to produce a high functional immune response, e.g., because they have never been exposed to the DMBs and the subject has thus never produced DMBs, or because the disease associated with the DMBs is under control or in remission. In this case, it is unlikely that the patient has or is developing the disease of interest. On the other hand, if one or more substances associated with a functional immune response is detected, then one can conclude that the subject does have or is developing the disease.

The final step in another embodiment of the method (the monitoring embodiment) also involves interpreting and then drawing a conclusion from the results. However, in this embodiment, the level of DMB that is detected is generally compared to one or more levels of DMB previously detected in the same subject (e.g., levels measured at earlier time points), and the results of the comparison are used to determine or to modify treatment protocols.

In alternative embodiments, the results are compared to those obtained from testing one or more control groups, e.g., age and/or gender matched controls. This embodiment is described in detail below.

A basic workflow for designing the DMB specific functional immune response assay is outlined below:

1. Sample collection from a subject. Samples could be e.g., peripheral blood, tissues or fluids originated from disease sites.
2. Subpopulation immune cell separation. This optional step may or may not be necessary or desirable, depending on the sample and the particular detection test that is to be carried out. In addition, defined subpopulations of immune cells could be used before or after the stimulation step to enhance the overall assay sensitivity and specificity, i.e., one or more subsets of immune cells may be separated and stimulated; or all immune cells may be stimulated but then one or more defined subsets are separated and analyzed for the presence of immune response-associated substances.
3. Stimulation. Stimulants include one or more than one type of DMBs. These DMBs could be any biomolecules such as peptides, proteins, antibodies, or nucleic acids and modified in the disease sites or through disease pathogenic mechanisms. The stimulation time and conditions such as temperature and CO₂ concentration are optimized using techniques that are known to those of skill in the art. In some embodiments, co-stimulants such as phytohaemagglutinin (PHA), lipopolysaccharide (LPS), antigens, superantigens such as Staphylococcal enterotoxin A (SEA) and Staphylococcal enterotoxin B (SEB), anti-CD3, anti-CD28, antibodies for forming immune complexes with DMBs, etc., are also added to the reaction.

Generally, in vitro stimulants may be added directly to test samples and then incubated at the desired temperature such as 37° C. for a suitable period of time (e.g., generally from about 0 to about 48 hours). The stimulation condition and time may be optimized so the appropriate signal to noise ratio is obtained. The stimulants may be prepared in solution, dried-down or lyophilized forms, and be supplied in the same collection tubes or in separate tubes. Several assay controls may be included and run side by side with the test samples. In addition, negative controls are generally included, e.g., the assay without antigen (DMB) stimulation or with non-specific antigens. Positive controls might be the assay plus a mitogen such as PHA, LPS, antigens, superantigens such as SEA and SEB, anti-CD3, etc.

4. Measurement of a functional response to DMB exposure.

For both diagnosis and monitoring applications, single or multiplex functional immune response(s) can be measured after sample stimulation by the antigen (the DMB). Such measurements may detect one particular molecule or more than one molecule, e.g., a group or panel of molecules may be detected. Further, the detected molecules may be produced specifically and exclusively in response to a particular DMB or type of DMB, or they may be produced non-specifically in response to several different DMBs, i.e., they may be general indicators of immune cell activation, examples of which include but are not limited to intracellular ATP. These measurements could detect, for example, interferon-gamma (IFN-γ), cytokine profiles or panels of markers, intracellular ATP concentration or other cell functions as the result of antigen specific cell immune activation, etc. Further, profiles or panels of markers from two different cell types from the same patient may be compared, e.g., T helper cells vs T regulatory cells, or T regulatory cells vs B regulatory cells, etc. Alternatively, one or more subpopulations of immune cells such as CD4+ or CD8+ may be used in the assay, or specific profiles associated subpopulations of immune cells may be measured.

In some embodiments, interpretation of the results obtained using the assays and methods of the invention are facilitated by provision of a “scale” or “rating system” based on results obtained with control subjects. For example, the quantity or level (i.e., the measured or detected amount) of a substance produced by stimulated cells may be compared, e.g., on a scale of 1 to 10, with the amount that is typically or on average detected in control subjects who do not have the disease being e.g., 2 or less, and the amount for persons who have advanced disease being e.g., 8 or more. Other intermediate disease stages that increase in severity may be assigned numeric values of e.g., 3-7, with 3 being the mildest response and 7 being the strongest of those. Such values may also be used as target values for treatment, e.g., for a patient who starts with a response level of 6 or 7, a reasonable target range to attain by using medical treatment might be preferably less than 2 if possible, if no side effects are experienced from the treatment, or perhaps only 2-3, if the benefits of treatment must be weighed against side effects. Those of skill in the art will recognize that there are many possible permutations of such numeric reference systems and many ways of expressing them, (e.g., 1-100, using decimals e.g., 1.0 to 10.0, or percentages, etc., including “all or nothing” binary systems where a simple “yes” or “no” answer is provided to the question: Does this patient have this disease?) and all such expression systems are intended to be encompassed by the present invention. The use of such scales may facilitate automation of the assays of the invention. The scale of results may also be expressed in the format of a ratio in which the numerator is the measured response (e.g., cytokine) and the denominator is a unit of measurement of the sample (e.g., volume, # of cells, per cell, etc.).

The overall workflow from sample collection, stimulation and cell function measurement may be adapted to an automated platform(s). The isolation/selection of blood cell subpopulations either before or after antigen stimulation might be necessary or desirable for obtaining or to maximize specific and reproducible functional immune responses.

In an exemplary embodiment, the invention provides a method of prognosing or diagnosing rheumatoid arthritis (RA) or, alternatively, of monitoring therapeutic efficacy of drugs used to treat RA, in a subject in need thereof. Both types of methods are carried out by detecting whether or not the immune system of the subject has been primed to specifically recognize endogenous citrullinated peptides (CPs), a hallmark of RA. The methods involve the steps of obtaining a biological sample from the subject, adding CPs to the sample, and detecting the presence of at least one molecule associated with a functional immune response to the CPs (e.g., IFN-γ, cytokines, intracellular ATP, etc.) or specific response profiles of these molecules. In some embodiments, immune stimulants are also added to the sample, together with the CPs. In other embodiments, the CPs are cyclic CPs (CCPs). In some embodiments, positive immune stimulants such as SEA, SEB or PHA are used, and non specific peptide or uncitrullinated proteins are added for controls

The CPs that are used in the practice of the invention generally comprise from about 1 to about 50 amino acid residues, usually from about 5 to 30 residues, and more often from about 10-25 residues, e.g. 11, 12, 13, 14, 15, 16, 17, 18, 19 or 20 residues. Alternatively, peptides/polypeptides of such lengths which contain one or more CPs as described herein within their primary sequence are also contemplated. Further, one or more than one type of CP may be added to the sample, i.e., a pool of CPs, of differing chemical compositions may be used. For example, one or more than one residue in the various CPs is citrullinated, and citrullinated residues may be located at any position within the peptides. Sequences of these stimulating CPs may not be necessary to be any sequence in nature expressed proteins but rather be capable to induce disease specific immune response. The identity of the non-citrullinated residues that are present in the peptide may vary, and may be any of those of the 20 common residues, or any known modification thereof, or any of the less common amino acids (2-aminoisobutyric acid, lanthionine, various D-amino acids, etc). Further, amino and/or carboxyl termini modifications (e.g., by protective or blocking groups, various tags or labels, etc.) may also be present. The CPs that are added may be identical to one another, or may vary in chemical composition, i.e. a pool of peptides may be utilized.

The invention also encompasses antibodies to the peptides and proteins disclosed herein. Those of skill in the art are familiar with antibodies and methods for preparing them. For example, an isolated peptide can be used as an immunogen to generate antibodies using standard techniques for polyclonal and monoclonal antibody preparation. A peptide immunogen typically is used to prepare antibodies by immunizing a suitable subject, (e.g., rabbit, goat, mouse or other mammal) with the immunogen. An appropriate immunogenic preparation can contain, for example, recombinantly expressed peptides or polypeptides/proteins comprising the peptide sequences disclosed herein, or a chemically synthesized peptide, polypeptide or protein. The preparation can further include an adjuvant, such as Freund's complete or incomplete adjuvant, or another immunostimulatory agent. Immunization of a suitable subject with an immunogenic preparation induces a polyclonal antibody response.

If desired, the antibody molecules can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497), or more recent human B cell hybridoma techniques (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds the immunogen. Moreover, the ordinarily skilled worker will appreciate that there are many variations of such methods which also would be useful. The invention also encompasses hybridoma cells and immortal cell lines generated in this manner.

Alternatively, to prepare monoclonal antibody-secreting hybridomas, a monoclonal antibody can be identified and isolated by screening a recombinant combinatorial immunoglobulin library (e.g., an antibody phage display library). Kits for generating and screening phage display libraries are known in the art and commercially available

The term “antibody” as used herein refers to immunoglobulin molecules and immunologically active portions of immunoglobulin molecules i.e., molecules that contain an antigen binding site which specifically binds (immunoreacts with) an antigen. Examples of immunologically active portions of immunoglobulin molecules include F(ab) and F(ab′)₂ fragments which can be generated by treating the antibody with an enzyme such as pepsin. Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art. All such antibodies and/or fragments thereof, and compositions comprising the same, are herein encompassed.

An antibody of the invention (e.g., monoclonal antibody) can be used to isolate or detect the citrullinated peptides described herein, and/or polypeptides or proteins comprising the peptides, using standard techniques, such as affinity chromatography or immunoprecipitation. An antibody can facilitate the purification or detection of the peptides, polypeptides and proteins disclosed herein, e.g., in a cellular lysate or cell supernatant or after chemical or recombinant synthesis. The antibodies can also be used diagnostically to monitor peptide/protein levels (e.g., in synovial fluid, blood, serum, etc.) as part of a clinical testing procedure, e.g., to, for example, diagnose a disease, or to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive tags include ¹²⁵I, ¹³¹I, ³⁵S, ³H, etc.

### Applications for the DMB Assay:

The present invention may be used for many applications, including but not limited to the following:

### Diagnostic Assays

The assays and methods described herein may be used either as stand alone assays or in combination with other clinical criteria or clinical measurements for the definitive diagnosis (e.g., as confirmation) of a disease of interest. Exemplary clinical measurements include but are not limited to scores on a scale of joint pain, etc. Such assays may be carried out, for example, with persons exhibiting symptoms of a disease, and may corroborate other diagnostic measures. In addition, such diagnostic assays may encompass prognostic assays, i.e., the assays may be used to predict the likelihood that an individual will develop symptoms of a disease of interest, even if they do not, at the time of the assay, have any symptoms. For example, individuals with a family history of a particular disease, or with a known genetic mutation that statistically predisposes individuals to the development of a disease, may benefit from an assessment as described herein. While a negative result would not necessarily rule out future development of the disease, a positive result would alert a subject to the presence of a risk of disease development and/or of the presence of incipient disease, enabling health care professionals to prescribe prophylactic treatment which would slow or even prevent disease development. In some embodiments, the invention also encompasses methods for developing or modifying treatment protocols for patients who have or are suspected of having a particular disease. Such methods include carrying out the assay of the invention and, depending on the outcome, initiating or adjusting a suitable treatment program for the patient. In yet other embodiments, the assays are used to screen individuals who may have no prior indication that they may have one or more diseases of interest, but who wish to monitor their general health. Such patients may wish to establish a “baseline” with respect to several diseases of interest at a relatively early age, and to check whether, with time, they are developing or appear to be prone to developing one or more of the diseases or conditions of interest.

### Monitoring Assays

In other embodiments, the assay may be used for monitoring disease progression and/or therapeutic efficacy. Once a patient is definitively diagnosed with a disease, and especially after therapy has begun (e.g., the administration of drugs or other medications, or diet or exercise changes, etc.), the assays may be used to monitor or track the patient's progress and/or the status or progression or regression of disease. In some embodiments, a level of the DMB is determined prior to treatment or prior to administration of an agent or procedure that is used to treat the disease, and a second level of the DMB is measured after treatment or administration, and the two levels are compared. If the level of DMB decreases after treatment or administration, then the agent may be deemed to be efficacious, and treatment may be continued with the agent, or the amount or frequency of may be decreased, or even discontinued if lowered levels of DMB (or no DMB) are detected. Alternatively, if the level is unchanged or higher, it may be concluded that the agent is not effective and, for example, the amount of agent that is administered may be increased, or the agent may be administered more frequently, or a different agent may be used instead or in combination with the agent to treat the disease or condition. As will be recognized by those of skill in the art, such information would be extremely useful, e.g., in order to quickly determine whether or not a drug is having a desired effect or if the desired effect is occurring at a suitable level, within a suitable time frame, etc. Thus, the invention also encompasses methods for developing and modifying treatment protocols for patients who have and are being treated for a particular disease. Such methods include carrying out the assay of the invention and, depending on the outcome, modifying or adjusting the patient's treatment plan. For example, the amount of a medication may be increased, decreased, or a medication may be discontinued, or discontinued in favor of using a different medication, or other therapies may be added to the protocol, etc. Such adjustments may be carried out in conjunction with other assays or evaluation tools, e.g., other tests such as blood tests, x-rays, MRI scans, reports from the patient, observations by trained personnel, etc. Patients may be monitored on a regular basis at predetermined time intervals, e.g., weekly, monthly, every few months, yearly, etc., or as needed, e.g., when symptoms begin to change, or when other factors that may impinge on treatment outcome are identified, etc.

### Rheumatoid Arthritis as an Illustrative Example

In some embodiments, the invention may be applied to the prognosis, diagnosis, and monitoring of RA. Recent discoveries have shown that post translational modification (PTM) of protein plays a significant role in autoimmune diseases. For example, in rheumatoid arthritis (RA), citrullination of native proteins such as vimentin, fibrin, histone and filaggrin has been found to occur. Citrullinated proteins and peptides (CPs) have been detected in synovial fluids and blood samples of RA patients, and anti-CCP autoantibodies (also called anti citrullinated protein antibody, or ACPA) have been detected in about 60-75% of RA patients with remarkably high specificity (96-98%). Significantly, anti-CCP autoantibodies have been detected as early as up to 15 years prior to the onset of disease, and several studies support the conclusion that the presence of anti-CCPs is of value in predicting the future development of RA in asymptomatic individuals.

As a result, there is much interest in the use of anti-CCPs, or ACPA, as an early biomarker for RA, and quantification of anti-CCP antibodies in RA patients has been suggested as a means of predicting disease damage and assessing treatment efficacy. In fact, several in vitro diagnostic (IVD) assays for detecting anti-CCP antibodies in serum or plasma samples have been developed and have received FDA clearance. Although anti-CCP assays display a high specificity (approximately 90% to 96%), significant numbers of RA patients never develop anti-citrullinated protein antibodies. Clinical sensitivities for anti-CCP assays from published reports range from 60 to 75%, approximately. Furthermore, there is lack of a consistent correlation between the titers of ACPA and disease activities/symptoms. The current invention detects the immune responses of patient's immune cells against DMBs rather than autoantibodies only. In diseases such as RA, type 1 diabetes and celiac disease, DMBs, not autoantibodies, may occur in the early disease progression phase. It is known that immune responses, such as the increased rate of secretion and synthesis of various cytokines induced by the presence of DMBs, is a critical part of disease pathogenesis. Many new biological drugs such as TNFα inhibitors have been recently developed to block immune responses to DMBs. Measurement of immune responses against DMBs is a more effective diagnosis and monitoring tool for disease activities as well as for the treatment or predicators of therapies.

In some embodiments, the methods and assays of the present invention are used to diagnose, prognose, and track the treatment of RA using CPs as an immune system stimulant. Because the assay of the invention relies on interrogating functional responses of the immune system, and because CPs frequently appear prior to the emergence of readily observable disease symptoms, use of the methods and assays described herein permits early diagnosis of RA and/or of a predisposition to the development of RA. As such, the methods and assays may be used to predict or prognosticate the presence of RA in a patient. The methods and assays may, for example, be used in conjunction with other methods (e.g., the identification of gene sequences that predispose or are indicative of a high likelihood of developing RA), to confirm a diagnosis of RA, or to screen individuals identified as susceptible to RA (i.e., at high risk for developing) RA, e.g., as predicted by family history, etc.

In other embodiments, the methods/assays may be advantageously used to monitor the progress and/or efficacy of treatment protocols in persons already being treated for RA, By monitoring the levels of immune system activation as described herein, it is possible to determine whether or not a particular treatment (e.g., a drug or other type of therapy) is having the intended effect, whether disease symptoms at the immunological level are being held in check, reversed, not affected, or continuing to progress in spite of treatment, etc. Depending on the results that are obtained, the treatment plan for the individual may be adjusted or may be maintained as is, or a decision may be made to delay, accelerate or to otherwise modulate treatment based on the patient's status, risk factors, etc.

The foregoing Examples serve to further illustrate various embodiments of the invention, but should not be construed so as to limit the invention in any way.

### EXAMPLES

### Example 1

### DMB Specific Functional Immune Response Assay for Diagnosis and Prognosis of Rheumatoid Arthritis (RA)

Autoimmune diseases generally occur as a result of malfunctions in the normal human immune system. A healthy immune system helps protect the body from harmful substances such as bacteria, viruses, toxins, cancer cells, etc. In autoimmune diseases, the immune system apparently cannot tell the difference between antigens associated with these harmful foreign entities and the body's own tissues, and the immune cells respond by producing autoantibodies which attack the body's own tissues. More than 80 different autoimmune diseases have been identified and the treatment of such diseases is mainly through drugs to control (usually decrease) the immune response.

Autoantibodies have been successfully utilized in developing clinical diagnostic assays for autoimmune diseases. It has long been believed that autoantiboics are produced as a result of an aberrant immune response against autoantigens. However, at least in some cases, it has been found that the autoantigens themselves are chemically or structurally modified by the disease process, resulting in production of disease modified biomolecules (DMBs) which are not naturally present in the body under healthy conditions. Such DMBs are antigenic and the immune system responds to their presence, for example, by producing autoantibodies and/or by initiating other immune response changes, e.g., changes in the regulation of T-cells and other immune cells. Thus, it is the DMBs which elicit an immune response against the “self”.

Rheumatoid arthritis is one such exemplary autoimmune disease. Although the pathogenic causes of RA are not known, it is clear that RA is a chronic and systematic autoimmune disease. RA is characterized by synovial joint inflammation with progressive erosion of bone and cartilage, which often leads to joint misalignment, loss of mobility, and premature mortality. RA affects 0.5 to 1% of the world population and there are more than 2 million RA patients in the USA alone. It is estimated about 10 million doctor visits per year in the US are attributable to RA, and many RA patients need hospitalizations and/or extended care due to progression of the disease.

Recent clinical trials data has demonstrated that the use of disease-modifying anti-rheumatic drugs (DMARDS) at an early stage of RA can slow down the course of disease progression and thus reduce patients' pain and suffering. Thus, early and definitive diagnosis of RA is vital for the management of this disease in patients and for preventing irreversible joint and tissue damage. Unfortunately, the definitive diagnosis of RA, especially in the early stages of disease, is difficult. Criteria established by the American College of Rheumatology (ACR) provide the main guidelines for RA diagnosis. These criteria include clinical observations such as morning stiffness and swelling around joints, blood tests for rheumatoid factor, ESR (erythrocyte sedimentation rate), the presence of CRP (C-reactive protein) and X-ray changes in joints. However, ACR classification criteria are rarely recognized during the early stages of the disease since at that point, RA symptoms are similar to those of other types of arthritis. As a result, the diagnosis of RA typically does not occur until about 2-5 years after onset of the disease. Unfortunately, radiographic data shows that significant destruction of cartilage and bone in joints may occur within the first two years of disease onset, and this damage often leads to joint deformities over time.

The immune system of individuals with RA who are anti-CCP positive is primed for responses to antigens with CP epitopes. Blood samples which are withdrawn from RA patients contain lymphocytes with immunological responsiveness to citrullinated peptides. Therefore the in vitro addition of one or a plurality of mixed CP peptides rapidly stimulates the immune response in blood samples. The antigen induced, specific functional immune response can then be measured.

The proposed assay includes following discreet steps

Sample collection and process. Whole blood samples and/or disease site samples are collected in blood collection tubes containing anti coagulation reagents, or in other suitable tubes, e.g., Cell Preparation Tubes (CPT™), gene prep tubes, etc. Blood samples are kept in a condition which maintains cellular stability and without external contact with the cellular immune system. Alternatively, peripheral blood mononuclear cells (PBMCs) or other blood processed samples may also be used for the current invention.

### Stimulation and DMB Selection.

The DMB antigen used in the assay is generally selected from:

Single synthetic citrullinated peptide

Mixture of two or more synthetic citrullinated peptides

Proteins with citrulline residues in place of arginine

Digested peptide pools from citrullinated proteins

Co-stimulating reagents to improve the assay performance

### Measurement of Functional Immune Response:

Single or multiplex functional immune response(s) can be measured after samples are stimulated by antigen.

IFN-γ, IL6, or TNFα

Cytokine profiles

Intracellular ATP concentration

Various cell surface markers

Molecular profiles of changes (e.g., up or down regulation) in mRNA, miRNA and other transcriptional indicators

Other cell functions modified by antigen specific immune activation

### Example 2

### Design and Testing of Citrullinated Peptides for Detection of Rheumatoid Arthritis

### Peptide Design and Synthesis

Peptide sequences were selected from human vimentin, fibrinogen (alpha or beta chain), filaggrin and alpha enolase. Citrullinated forms of these proteins are known to be reactive with autoantibodies from patients suffering from rheumatoid arthritis. A total of 16 peptides in citrullinated or non-citrullinated forms were chemically synthesized (Table 1). The purity of these synthesized was at least 95%.

In order to evaluate cellular stimulation responses for these peptides and thus identify citrullinated peptides for use in the practice of the invention, peptides were designed based on a) the protein source of the peptides; b) the number of citrullinated residues per peptide (which ranged from 0 to 3); and c) the distribution pattern of citrulline residues in each peptide.

TABLE 1
DMB peptide sequences

| | | # of | |
|---|---|---|---|
| Peptide | Protein | citrulline | |
| name | sources | residues | Peptide sequences |
| DMB 1001 | Vimentin | 3 | VYAT[Cit]SSAV[Cit]L[Cit]SSVP |
| | | | (SEQ ID NO: 1) |
| DMB 1002 | Vimentin | 0 | VYATRSSAVRLRSSVP |
| | | | (SEQ ID NO: 2) |
| DMB 1003 | Fibrinogen | 1 | NEEGFFSA[Cit]GHRPLDKK |
| | beta chain | | (SEQ ID NO: 3) |
| DMB 1004 | Fibrinogen | 0 | NEEGFFSARGHRPLDKK |
| | beta chain | | (SEQ ID NO: 4) |
| DMB 1005 | Filaggrin | 1 | HQCHQEST[Cit]GRSRGRCGRSGS |
| | | | (SEQ ID NO: 5) |
| DMB 1006 | Filaggrin | 0 | HQCHQESTRGRSRGRCGRSGS |
| | | | (SEQ ID NO: 6) |
| DMB 1007 | Fibrinogen | 2 | GP[Cit]VVE[Cit]HQSACKDS |
| | alpha chain | | (SEQ ID NO: 7) |
| DMB 1008 | Fibrinogen | 0 | GPRVVERHQSACKDS |
| | alpha chain | | (SEQ ID NO: 8) |
| DMB 1009 | Alpha enolase | 2 | KIHA[Cit]EIFDS[Cit]GNPTVE |
| | | | (SEQ ID NO: 9) |
| DMB 1010 | Alpha enolase | 0 | KIHAREIFDSRGNPTVE |
| | | | (SEQ ID NO: 10) |
| DMB 1011 | Vimentin | 3 | GGVYAT[Cit]SSAV[Cit]L[Cit]SSVP |
| | | | (SEQ ID NO: 11) |
| DMB 1012 | Vimentin | 2 | GGVYAT[Cit]SSAVRL[Cit]SSVP |
| | | | (SEQ ID NO: 12) |
| DMB 1013 | Vimentin | 2 | GGVYATRSSAV[Cit]L[Cit]SSVP |
| | | | (SEQ ID NO: 13) |
| DMB 1014 | Vimentin | 1 | GGVYATRSSAV[Cit]LRSSVP |
| | | | (SEQ ID NO: 14) |
| DMB 1015 | Fibrinogen | 3 | GGV[Cit]GP[Cit]VVE[CIT]HQSACKD |
| | | | (SEQ ID NO: 15) |
| DMB 1016 | Fibrinogen | 3 | HSTK[Cit]GHAKS[Cit]PV[Cit]DCDD |
| | | | (SEQ ID NO: 16) |

*Cit: citrulline

### Identification of Specific Citrullinated Peptide Sequences

Experiment 1: Cytokine Production after Stimulation of PBMCs from RA Patients with Individual Peptides DMB 1001-DMB 1010

Experimental setting: Peripheral blood mononuclear cells (PBMCs) were prepared from freshly collected blood using a Ficoll gradient method. The cellular stimulation assay was performed by incubation of PBMCs with the individual peptides DMB 1001-DMB 1010 at a concentration of 10 μg/ml. Approximately 300,000 to 500,000 PBMC cells were used for each stimulation assay in a cell culture AIM V serum free medium (Life Technologies) within a 96 well round bottom plate. TNFα, IL6 and IL1b concentrations in culture supernatants were determined using the AlphaLisa® method after 20 hrs of incubation.

Results: The concentrations of TNFα in culture supernatants stimulated by peptide 1001 were 163.1 pg/ml, and 44.3 pg/ml from samples 1 and 2, respectively. Both values were significantly higher than the negative control wells (where peptides were not added). These higher concentrations of TNFα were not observed in wells in which other peptides were tested (Table 2A). The sequence of peptide 1002 is identical to that of peptide 1001 but is not citrullinated. Similarly, higher concentrations for IL6 and IL1b were also found in supernatants from cells stimulated using peptide 1001, but not in wells stimulated with peptide 1002 (Table 2B).

TABLE 2A
TNFα concentrations after peptide stimulation using PBMCs

TNFα concentration
(pg/ml)

| Peptide | Sample 1 | Sample 2 |
|---|---|---|
| 1001 | 163.1 | 44.3 |
| 1002 | 7.7 | 2.7 |
| 1003 | 4.1 | 2.2 |
| 1004 | 2.5 | 6.7 |
| 1005 | 2.1 | 3.6 |
| 1006 | 2.7 | 7.6 |
| 1007 | 4.3 | 4.6 |
| 1008 | 7.1 | 7.5 |
| 1009 | 5.0 | 6.4 |
| 1010 | 2.3 | 3.6 |
| NS | 1.5 | 0.7 |
| LPS | 1708.9 | 508.9 |

TABLE 2B
Cytokine concentrations in cell cultures using peptide 1001 vs. 1002

| Peptide | |
|---|---|
| 1001 | 1002 |

Sample

| | Sample 1 | Sample 2 | Sample 1 | Sample 2 |
|---|---|---|---|---|
| TNFα | 163.1 | 44.3 | 7.7 | 2.7 |
| IL6 | 664.7 | 320 | 0 | 0 |
| IL1b | 80 | 46 | 1 | 0 |

Experiment 2. Cytokine Production after PBMC Stimulation Using Individual Peptides from DMB 1011 to DMB 1016

Experimental setting: Peripheral blood mononuclear cells (PBMCs) were prepared from freshly collected blood using a Ficoll gradient method. The cellular stimulation assay was performed by incubating PBMCs with individual peptides DMB 1011 to DMB 1016. Approximately 300,000 to 500,000 PBMC cells were used for each stimulation assay in a cell culture AIM V serum free medium (Life Technologies) within a 96 well round bottom plate. Concentrations of TNFα and IL6 in culture supernatants were determined after 20 hrs incubation using the AlphaLisa® method.

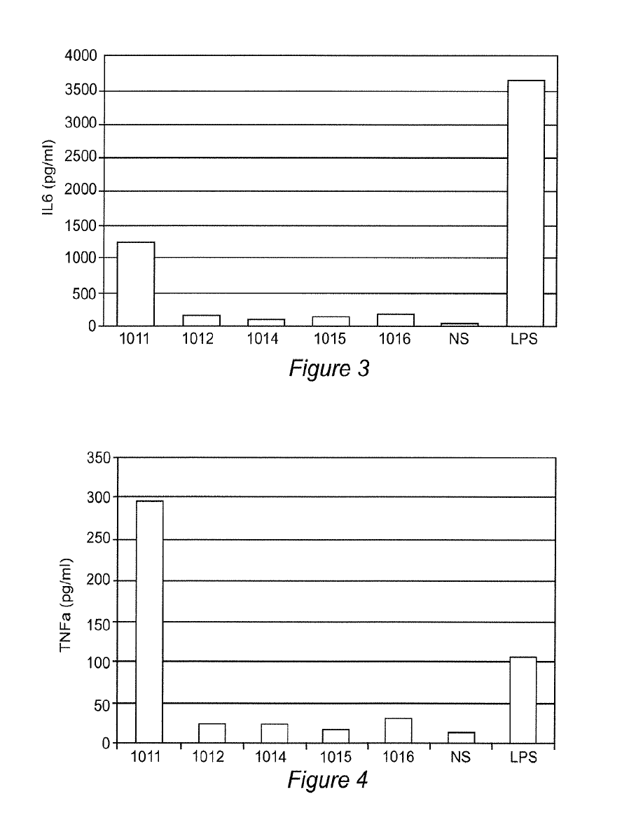

Results: Peptide DMB 1011 has the same sequence of DMB 1001 but adds two amino acids (Gly-Gly) to its N terminal. The addition of two amino acids improved the peptide solubility in water. Significantly higher concentrations of IL6 (FIG. 3) and TNFα (FIG. 4) were detected in cultures stimulated by DMB 1011 but not those stimulated with other peptides.

### Conclusion:

Data from Experiments 1 and 2 demonstrate that peptides DMB 1001 and DMB 1011 have a specific peptide consensus (motif) in citrullinated form that can stimulate PBMCs to secrete cytokines such as TNFα and IL6.

Both TNFα and IL6 levels in RA patients are elevated and have been the major drug targets for treatment of RA. The identification of the peptide motif contained within DMB 1001 and DMB 1011 makes it possible to identify, and/or confirm the identification of, individuals suffering from RA using the methods of the invention.

### Example 3

### Individual Variations of Stimulation Response Against DMB 1001

### Experimental Setting:

Peripheral blood mononuclear cells (PBMCs) were obtained from RA patients and prepared using a Ficoll gradient method. A cellular stimulation assay was conducted by incubating the PBMC cells with DMB 1001 at 10 μg/ml. Cells were incubated for 20 hrs at 37° C. and 5% CO₂ IL6 and TNFα concentrations in the culture supernatants were determined by using the AlphaLisa® assay.

### Results:

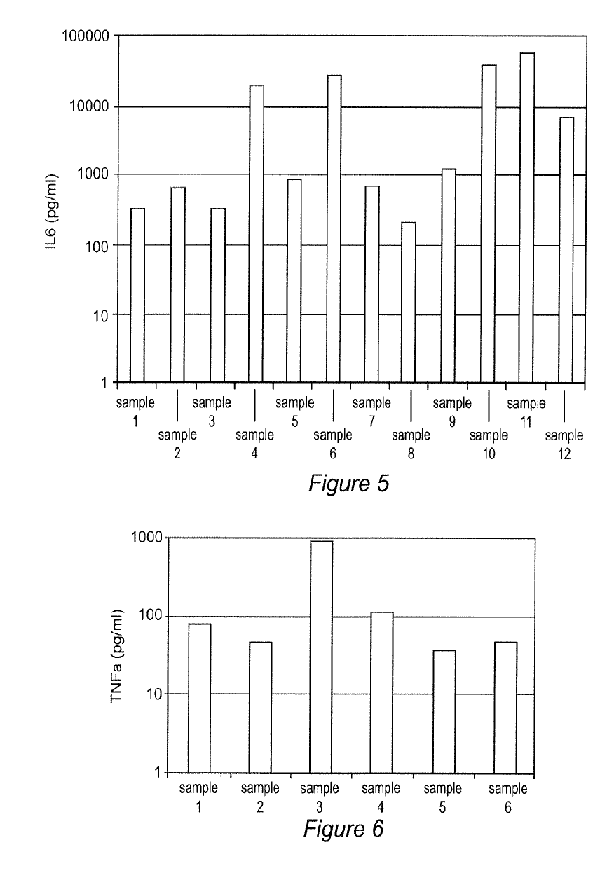

Upon stimulation with DMB 1001, IL6 concentrations in the culture media were in the range of approximately 100 to 60,000 pg/ml (FIG. 5). Among the 12 PBMC samples, 5 samples had a cytokine level greater than 1000 pg/ml. The data clearly showed that the cellular immune response to DMB 1001 varied significantly among individuals. This result correlated well with the clinical finding that IL6 levels in plasma found in RA patients were elevated but in a wide range.

Individual variations in response to stimulation with peptide 1001 were also observed when TNFα was measured (FIG. 6). As can be seen, a more than 10 fold difference in TNFα concentrations among 6 samples tested was detected.

Several proinflammatory cytokines such as TNFα and IL6 have been shown to be present at elevated levels in the synovial fluid and plasma of patients with RA. The introduction of biologics such as TNFα- and IL6-inhibitors has revolutionized the treatment of RA. One of critical challenges in the biologic therapy of RA patients is that up to 60% of patients do not respond to these drugs.

This cellular stimulation assay using DMB 1001 and/or DMB1011 provides a companion diagnostic assay for these drugs, and may provide an explanation regarding why some patients respond while others do not. Patients with high levels of TNFα and/or IL6 may be more likely to respond to therapy that inhibits these cytokines, whereas patients with low levels may be less likely to respond well and be treated successfully.

### Example 4

### Differential Cytokine Profiles after Cells Stimulated with DMB 1001

### Experimental Setting:

Freshly collected blood was obtained from one apparently healthy adult (AHA), one patient with multiple sclerosis (MS) and two RA patients. Peripheral blood mononuclear cells (PBMCs) were prepared from the blood samples using a Ficoll gradient method. Cellular stimulation assays were performed by incubating PBMCs with DMB 1001 at 10 μg/ml for 20 hrs at 37° C. and 5% CO₂ Cytokine concentrations in the culture supernatants were determined by using a Cytometric Bead Assay (CBA). A Cytokine Index was calculated using the formula: Index=100× (DMB 1001-NS)/(SEB-NS) where SEB is the positive control and NS is the negative control. SEB: Staphylococcal enterotoxin B. NS: non-stimulation.

Results:

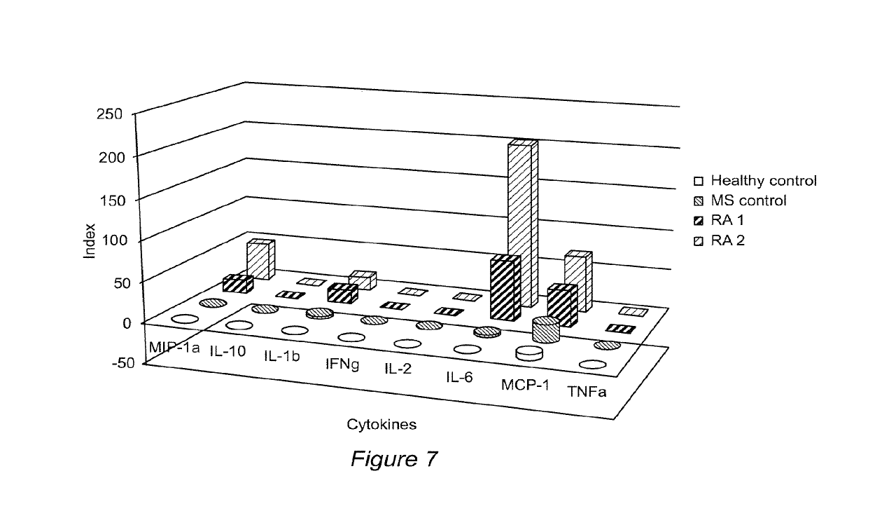

A total of 8 different cytokines were tested in this experiment and the index score for each cytokine was the calculated. The results (presented in FIG. 7) showed the cytokine profiles from the two RA patients were significantly different from those of the healthy and MS controls.

### DISCUSSION

### Rationales for Designing Citrullinated Peptides

The presence of autoantibodies against citrullinated proteins (ACPA) in the serum of RA patients has been utilized as an aid for the diagnosis and classification of RA in combination of other clinical observations and laboratory tests including numbers of joints involved, duration of disease, and the serum level of C-reactive protein (CRP). However, until the prior invention, it was not clear that what citrullinated proteins in vivo actually triggered the production of ACPA in patients. In commercial anti-CCP tests, artificial citrullinated peptides have been used for detecting ACPA in the patient serum instead of natural protein sequences, since the natural sequences were unknown.

Current literature suggests that potential autoantigens against ACPA include vimentins, fibrinogen, α-enolase and filaggrin. Among these proteins, vimentins, fibrinogen and α-enolase have been found in joints in significant amounts whereas filaggrin has not been detected in joints. However, since citrullinated filaggrin is reactive to ACPA found in RA patients, this protein was included in the present studies. As shown in Table 1 above, a total of 16 citrullinated and non citrullinated peptides were chemically synthesized and all peptides were purified to a minimum of 95% purity. The peptide sequences were derived from vimentin, alpha or beta chains of fibrinogen, filaggrin, and α-enolase.

In addition to choosing which proteins to study, another important aspect of designing peptides is the location of citrulline residues within peptides and the level of citrullination of arginine to citrulline. Although research in the field suggests that MHC class II-dependent activation of adaptive immunity should be the working model for RA pathogenesis, clearly MHC II independent mechanisms are also an integral part of the immune response and must play an important role in RA disease progression. In addition, there are at least 4 different PADI (peptidylarginine deiminase) enzymes capable of citrullination, and PADI4 has been reported to be involved in the citrullination process specifically involved in RA. The substrate specificity of PADI enzymes, sources of these enzymes and accessibility to intracellular proteins such as vimentins are unresolved and under active investigation.

The number of citrulline residues within the synthesized peptides ranges from 0 to 3. The stimulation response from DMB 1001 which has 3 citrulline residues, as measured by two important cytokines related to RA, are at least several fold higher than DMB 1002, which has the same sequence but was not citrullinated (see FIGS. 3 and 4). Stimulation responses were greatly diminished in peptides with the same sequence but having only one or two citrullines (DMB 1012, 1013 and 1014). These data demonstrate that full stimulation requires all three citrulline residues.

The next question addressed was whether 3 citrulline residues within the peptides are sufficient to stimulate immune cells. DMB 1014 and DMB 1015 are sequences derived from fibrinogen. Both peptides contained 3 citrulline residues with a similar pattern of location as DMB 1011. Data clearly showed that these fibrinogen based and citrullinated peptides failed to stimulate cells to produce TNF and IL-6. Other citrullinated peptides derived from filaggrin and alpha-enonlase also did not stimulate blood cells. This experimental evidence demonstrates that both protein sequence and citrullination patterns are required for stimulation.

Due to citrullination, in which positively charged arginine is converted to neutral citrulline, DMB 1001 is not water soluble, and has to be prepared in DMSO or another similar solvent. DMB 1011 was designed to contain the entire sequence of DMB 1001 plus two additional amino acids (GlyGly) which were added to its N terminus to make the peptide water soluble. When DMB 1011 was tested, the results showed that this water soluble peptide displayed stimulation effects on immune cells similar to those observed with DMB 1001.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of determining whether or not a subject has or is at risk of developing a disease or condition of interest, comprising the steps of
obtaining at least one disease modified biomolecule (DMB) that is identified as being present in biological samples from one or more subjects in a pool of subjects with the disease of interest, wherein the DMB does not appear or the DMB level is significantly lower in biological samples from one or more control subjects who do not have the disease or condition of interest;
obtaining a biological sample from a subject that has or may have the disease or condition of interest;
exposing the biological sample or cells from the biological sample to the DMB;
detecting at least one functional immunological response in the biological sample; and, if at least one functional immunological response is detected, then
concluding that the subject has or is at risk of developing the disease or condition of interest.

2. The method of claim 1 wherein said DMB is present in at least 50% of biological samples obtained from said pool of subjects.

3. The method of claim 1, wherein said step of concluding includes a step of comparing results obtained in said detecting step with results obtained in at least one of:
i) control subjects who do not have the disease or condition of interest;
ii) control subject who do have the disease or condition of interest;
iii) patients with the disease or condition of interest who are successfully responding to treatment; and
iv) patients who are positive for the presence of the at least one DMB but who have not developed other symptoms of the disease or condition of interest

4. The method of claim 1, wherein said at least one functional immunological response is selected from the group consisting of production of expression products of one or a plurality of genes; production of one or a plurality of mRNA molecules; production of one or a plurality of microRNAs (miRNAs); production of one or a plurality of proteins; and production of one or more of ATP, cytokines, interferon-gamma (IFN-γ), glucose, and nicotinamide adenine dinucleotide (NADH).

5. The method of claim 1, wherein said disease or condition of interest is rheumatoid arthritis (RA) and said DMB is a citrullinated peptide or protein (CP).

6. The method of claim 5, wherein said CP is or comprises a citrullinated amino acid sequence VYAT[Cit]SSAV[Cit]L[Cit]SSVP (SEQ ID NO: 1), or a functional variant thereof.

7. The method of claim 5, wherein said CP is or comprises a citrullinated amino acid sequence GGVYAT[Cit]SSAV[Cit]L[Cit]SSVP (SEQ ID NO: 11), or a functional variant thereof.

8. A method of monitoring the efficacy of treatment of a disease or condition of interest in a patient in need thereof, comprising the steps of
i) obtaining at least one disease modified biomolecule (DMB) that is identified as being present in biological samples from one or more subjects in a pool of subjects with the disease of interest, wherein the DMB does not appear or the DMB level is significantly lower in biological samples from one or more control subjects who do not have the disease or condition of interest;
ii) obtaining a biological sample from a subject that has and is receiving treatment for the disease or condition of interest;
iii) exposing the biological sample or cells from the biological sample to the DMB;
iv) detecting and/or quantifying at least one functional immunological response in the biological sample; and, based on results obtained in said detecting and/or quantifying step
v) determining whether or not said treatment is efficacious.

9. The method of claim 8 wherein said DMB is present in at least 50% of biological samples obtained from said pool of subjects.

10. The method of claim 8, further comprising the steps of
repeating steps i)-iv) for said patient at each of a plurality of successive, spaced-apart time intervals; and
comparing results obtained at said plurality of successive, spaced-apart time intervals in order to determine whether or not said treatment is efficacious.

11. The method of claim 8, wherein said at least one functional immunological response is selected from the group consisting of: production of expression products of one or a plurality of genes; production of one or a plurality of mRNA molecules; production of one or a plurality of microRNAs (miRNAs); production of one or a plurality of proteins; and production of one or more of ATP, cytokines, interferon-gamma (IFN-γ), glucose, and nicotinamide adenine dinucleotide (NADH).

12. The method of claim 8, wherein said disease or condition of interest is rheumatoid arthritis (RA) and said DMB is a citrullinated peptide or protein (CP).

13. The method of claim 12, wherein said wherein said CP is or comprises a citrullinated amino acid sequence VYAT[Cit]SSAV[Cit]L[Cit]SSVP (SEQ ID NO: 1), or a functional variant thereof.

14. The method of claim 12, wherein said CP is or comprises a citrullinated amino acid sequence GGVYAT[Cit]SSAV[Cit]L[Cit]SSVP (SEQ ID NO: 11), or a functional variant thereof.

15. A composition comprising
a citrullinated peptide or protein comprising one or both of
i) a citrullinated amino acid sequence VYAT[Cit]SSAV[Cit]L[Cit]SSVP (SEQ ID NO: 1) or a functional variant thereof, and
ii) a citrullinated amino acid sequence GGVYAT[Cit]SSAV[Cit]L[Cit]SSVP (SEQ ID NO: 11) or a functional variant thereof; and
a physiologically acceptable carrier.

16. The composition of claim 15, wherein said citrullinated peptide is or comprises VYAT[Cit]SSAV[Cit]L[Cit]SSVP (SEQ ID NO: 1) or a functional variant thereof.

17. The composition of claim 15, wherein said citrullinated peptide is or comprises GGVYAT[Cit]SSAV[Cit]L[Cit]SSVP (SEQ ID NO: 11) or a functional variant thereof.
